(19) United States
(12) Patent Application Publication
(10) Pub. No.: US 2002/0120008 A1
(43) Pub. Date: Aug. 29, 2002

(54) Life extension of drosophila by a drug treatment

(76) Inventors: Seymour Benzer, San Marino, CA (US); Kyung-Tai Min, Rockville, MD (US)

Correspondence Address: MANDEL & ADRIANO, 35 NORTH ARROYO PARKWAY, SUITE 60, PASADENA, CA, 91103, US

(21) Appl. No.: 09/895,141

(22) Filed: Jun. 29, 2001

Related U.S. Application Data
(60) Provisional application No. 60/215,401, filed on Jun. 29, 2000.

(51) Int. Cl. A61K 31/22; A61K 31/192; A61K 31/16

(52) U.S. Cl. 514/547; 514/570; 514/625

(57) ABSTRACT

The present invention provides methods for extending the life span of a subject and methods for inducing molecular changes within a whole organism that are responsible for the extended life span of the organism; therefore, providing a whole organism system to identify molecules involved in the ageing process.

This invention was made with Government support under NIH Grant No. AG 16630 and NSF Grant No. MCB 9907939. The Government has certain rights in this invention.

This application claims the priority of provisional patent application U.S. Ser. No. 60/215,401, filed Jun. 29, 2000, the contents of which are incorporated by reference in their entirety into the present application.

Throughout this application various publications are referenced. The disclosures of these publications in their entireties are hereby incorporated by reference into this application in order to more fully describe the state of the art to which this invention pertains.

### FIELD OF THE INVENTION

The present invention relates to the field of ageing and extending the life span of organisms using agents that inhibit histone deacetylase such as inhibitor of histone deacetylases.

### BACKGROUND OF THE INVENTION

The mechanisms of ageing or life span extension are daunting in their complexity (Arking, R. Biology of Aging: observations and principles (Sinauer, ed. 2 Sunderland, 1998), yet recent studies show that they can be understood at the molecular level. Long-lived forms of C. elegans, yeast, and Drosophila are developed by genetic manipulation, and provide whole organism models for studying ageing.

In Drosophila melanogaster a mutation in the Drosophila G protein-coupled receptor, methuselah, not only increases life span, but enhances resistance to stress caused by starvation, high temperature, and the free radical generator paraquat (Lin, Y. J., Seroude, L. & Benzer, S. Extended life-span and stress resistance in the Drosophila mutant methuselah Science 282, 943-946 (1998)). Another mutant, Indy, involving a sodium dicarboxylate cotransporter, also dramatically increases the life span of Drosophila without apparent weakening in fecundity or behavior (Rogina, B., Reenan, R. A., Nilsen, S. P. & Helfand, S. L. Extended life-span conferred by cotransporter gene mutations in Drosophila Science 290, 2137-2140 (2000)).

There have been various reports of global molecular changes associated with ageing, by comparing tissues from young and old animals (Lee, C. et al. Gene expression profile of aging and its retardation by caloric restriction Science 285, 1390-1393 (1999); Shelton, D. N. et al., Microarray analysis of replicative senescence Current Biology 9, 939-945 (1999); Zou, S., Meadows, S., Sharp, L., Jan, L. Y. & Jan, Y. N. Genome-wide study of aging and oxidative stress response in Drosophila melanogaster Proc. Natl. Acad. Sci. U.S.A. 97. 13726-13731 (2000)), but it is difficult to determine which events are directly involved in the ageing process.

Genetic studies in the fruit fly Drosophila melanogaster have identified conserved developmental pathways between vertebrate and invertebrate organisms that suggest a closer evolutionary relationship between vertebrate and invertebrate organisms than what had been earlier accepted. Disruption within these conserved molecular pathways result in similar defects in both vertebrates and invertebrates. Thus the utility of Drosophila as a model organism for the study of human disease is now well documented (Reiter et al. 2001, Genome Research, 11:1114-1125). In addition, Drosophila provides an excellent whole organism model system to identify molecules that cause molecular alterations involved in a complex biological processes such as ageing.

There is a need for methods or assays that induce molecular changes within a whole organism so that one can determine which molecular changes are responsible for the extended life span of the organism and whole organism systems to identify molecules involved in the ageing process.

### SUMMARY OF THE INVENTION

The present invention provides methods for extending the life span of a subject by administering an inhibitor of histone deacetylase (e.g. butyric acid derivative) to the subject, in an amount effective to extend the life, of the subject.

In addition, the present invention provides methods for identifying molecules that extend the life span of a subject. This method is carried out by administering to the subject a molecule of interest and an inhibitor of histone deacetylase.

Also, the present invention provides methods for identifying molecular alterations in a subject administered an inhibitor of histone deacetylase to induce ageing or extended life span duration. The identification of a molecular alteration in the subject is done by determining the presence, level and/or modification of nucleic acids or proteins in the subject and comparing that with molecular alterations in a subject not administered or exposed to the inhibitor of histone deacetylase. The molecular alteration in the subject exposed to the inhibitor of histone deacetylase which is different from, (i.e., not found in) the subject not exposed to the inhibitor of histone deacetylase are the molecular alterations effected by the inhibitor of histone deacetylase. An example of a molecular alteration includes, but is not limited to the induction of genes.

In addition, the present invention further provides methods to identify molecular alterations in a subject that has been administered an inhibitor of histone deacetylase and a molecule of interest. This method is carried out by administering the molecule of interest and an inhibitor of histone deacetylase to a subject. Identification of molecular alterations in the subject exposed to both the molecule of interest (e.g. a test molecule) and the inhibitor of histone deacetylase are done by comparing the presence, level and/or modification of nucleic acids or proteins in the subject with the molecular alteration in a subject exposed to a inhibitor of histone deacetylase but not the molecule of interest. The molecular alteration in the subject receiving both the molecule and the inhibitor of histone deacetylase which is different from the subject receiving the inhibitor of histone deacetylase but not the molecule of interest identifies the molecular alteration effected by the molecule of interest.

### BRIEF DESCRIPTION OF THE FIGURES

FIG. 1 are graphs depicting the results of administering PBA to Drosophila as described in Example 1, infra.

FIG. 2 are graphs depicting the effect of administrating PBA to Drosophila at different times during development as described in Example 1, infra.

FIG. 3 are bar graphs depicting activity level and resistance to stress in Drosophila having been administered PBA as described in Example 1, infra.

FIG. 4 is a western blot that shows the effect of administering PBA to Drosophila on acetylation of histones H3 and H4 as described in Example 2, infra.

FIG. 5 shows the induction or repression of transcription of various genes in Drosophila administered PBA and verified by RT-PCR, as described in Example 2, infra.

Table 1 is a comparison of weight, size and reproductive ability of Drosophila raised on food with and without 10 mM PBA for 10 days at 25° C., as described in Example 1, infra.

Table 2 is a complete list of genes induced or repressed in flies fed PBA at 29° C., based on large differences in hybridization on membranes, clone ID refers to the GenBank identification number, as described in Example 2, infra.

Table 3 is a partial list of genes strongly induced or repressed in flies after 10 days of feeding with PBA at 29° C., based on large differences in hybridization on membranes, clone ID refers to the GenBank identification number, as described in Example 2, infra.

Table 4 is a list of primer sequences used for RT-PCR to confirm hybridization spots on the microarray, as described in Example 2 infra.

### DETAILED DESCRIPTION OF THE INVENTION

### Definitions

As used in this application, the following words or phrases have the meanings specified.

As used herein, “inhibitor of histone deacetylase” includes, but is not limited to, enzymes such as Trichostatin A (TSA), trapoxin, sodium butyrate, and suberoylanilide hydroxamic acid (SAHA) and inhibitor of histone deacetylases and butyric acid derivative such as isobutyramide, monobutyrin, tributyrin, 2-phenylbutyric acid, 3-phenylbutyric acid, 4-phenylbutyric acid, phenylacetic acid, cinnamic acid, alpha-methyldihydrocinnamic acid, 3-chloropropionic acid or vinyl acetic acid and salts thereof.

As used herein, “a salt of inhibitor of histone deacetylases” include, but are not limited to, mineral or organic acid salts of basic residues such as amines; alkali or organic salts of acidic residues such as carboxylic acids, and the like. Pharmaceutically acceptable salts include, but are not limited to, hydrohalides, sulfates, methosulfates, methanesulfates, toluenesulfonates, nitrates, phosphates, maleates, acetates, lactates and the like. Lists of suitable salts are found in Remington's Pharmaceutical Sciences, 17th ed., Mack Publishing Company, Easton, Pa., 1985, p. 1418, the disclosure of which, is hereby incorporated by reference in its entirety. Pharmaceutically acceptable salts also include amino acid salts such as arginine and lysine salts.

The “molecules of interest” for use in the methods of the invention include any organic molecule or chemical compound (naturally occurring or non-naturally occurring), such as a biological macromolecule (e.g., nucleic acid, protein, non-peptide, or organic molecule), or an extract made from biological materials such as bacteria, plants, fungi, or animal (particularly mammalian) cells or tissues, protein or protein fragment. Molecules of interest are evaluated for the potential to act as inhibitors or activators of a biological process or processes, e.g., to act as agonist, antagonist, partial agonist, partial antagonist, antineoplastic agents, cytotoxic agents, inhibitors of neoplastic transformation or cell proliferation, and cell proliferation-promoting agents. The activity of the molecules of interest may be known, unknown or partially known. Essentially any chemical compound can be used as a molecule of interest in the methods or assays of the invention, although compounds that can be dissolved in aqueous or organic (especially DMSO-based) solutions are preferred. It will be appreciated by those of skill in the art that there are many commercial suppliers of chemical compounds, including Sigma Chemical Co. (St. Louis, Mo.), Aldrich Chemical Co. (St. Louis, Mo.), Sigma-Aldrich (St. Louis, Mo.), Fluka Chemika-Biochemica Analytika (Buchs, Switzerland), and the like.

As used herein “extending the life span” means increasing the length of life of a subject beyond the normal expected life span of the subjector surviving longer than a normal subject. For example, extending the life span of the subject can be determined by comparing the life spans of (1) the subject administered with an inhibitor of histone deacetylase and (2) a second subject that is not administered with the inhibitor of histone deacetylase. If the subject exposed to the inhibitor of histone deacetylase exhibits a longer life span than that of the second subject that is not exposed to the inhibitor of histone deacetylase then this is indicative that the inhibitor of histone deacetylase effects life span extension.

As used herein “4-phenylbutyric acid” (PBA) means any soluble form of 4-phenylbutyric acid. All salt derivatives of PBA are considered to have the same function as the soluble PBA.

As used herein “subject” is used to represent invertebrate and vertebrate organisms. Examples of invertebrate subjects include, but are not limited to, insects (such as Drosophila), nematodes (e.g., Caenorhabditis (such as C. elegans)), and examples of vertebrate subjects include, but are not limited to amphibians (such as Xenopus), humans, equines, porcines, bovines, murines, canines, felines, or avians.

As used herein “a mutant subject” is a viable organism that has a genotype or phenotype that is distinguishable from its wild-type genotype or phenotype.

As used herein “wild-type Drosophila” refers to a control strain that exhibits prototypical Drosophila behavior.

As used herein “non-wild type Drosophila” refers to a viable Drosophila with a mutation, that shows a detectable phenotype or genotype which is different from the wild-type phenotype or genotype. Also referred to as a mutant Drosophila.

As used herein “strain” means a wild-type organism of one species with a different genotype from another wild-type organism of the same species that can mate with each other and produce viable offspring that can reproduce. Examples of strains of Drosophila melanogaster are double eleven eighteen (w¹¹⁸) and Canton-S.

As used herein “molecular alterations” refers to, but is not limited to, changes in protein levels, post-translational modifications of proteins, RNA expression levels, expression of alternative splice variants, and changes in DNA modifications such as, but not limited to methylation. Molecular alterations may encompass other known molecular alterations such as protein phosphorylation.

As used herein, “molecular alterations” refers to, but is not limited to, changes in RNA transcript levels and changes in protein expression levels. Changes in RNA transcript levels includes: changes in transcription of a gene sequence (e.g, transcription on or off); changes in the rate of transcription (e.g., induced transcription or constitutive transcription); changes in transcription start and stop sites; changes in transcript splicing, including changes in the rate of splicing and alternative splice sites; changes in transcript degradation, including changes in degradation of a particular transcript and changes in degradation rate; and changes in transportation of transcripts to sites within the cell, such as transport to the endoplasmic reticulum or other sites in the cytoplasm. Changes in protein expression levels includes: changes in translation of a particular RNA transcript (e.g., translation on or off); changes in the rate of translation (e.g., induced or constitutive translation); changes in post-translational modification, including peptide cleavage (e.g., cleavage site or rate of cleavage, glycosylation, phosphorylation, and acetylation); changes in protein folding which may or may not involve other cellular factors such as other proteins or RNA molecules; and changes in protein degradation. Additionally, changes in protein expression levels includes changes in protein transport, including transport to: the cell surface for export (e.g., secreted proteins); transport to the cell surface (e.g, cell receptors); transport to organelles, such as nucleus, mitochondria, chloroplast, Golgi, endoplasmic reticulum, ribosomes, vacuoles, lysosomes, peroxisomes, nucleolus, and centrioles. Changes in protein transport also includes transport to other site of the cell, including the plasma membrane, organellar membranes, cytoskeleton, ciliar, and flagella.

As used herein “changes in the RNA” refers to changes in RNA expression pattern, for example, RNA induction or RNA repression. Expression of alternative splice variants is also considered to be a change in the RNA expression pattern in a subject.

As used herein “changes in protein” refers to changes in protein translation such as, altered levels of protein translation or protein modifications. Examples of protein modifications are acetylation and phosphorylation.

As used herein “normal function” refers to the behavior of the organism. Examples of behavior that are considered part of an animal's normal function are reproductive behaviors, which include the health of the progeny and the ability of the progeny to reproduce. Other normal behaviors include but are not limited to sexual or mating behaviors, resistance to stress, ability to learn, ability to improve and/or access memory, level and vigor of activity such as walking, climbing, flying, or swimming.

In order that the invention herein described may be more fully understood, the following description is set forth.

The invention provides methods for chemically inducing an extended life span of a subject and a molecular analysis that identifies the genes, RNAs, and proteins effected by the chemically induced extension of life span of the subject.

In an embodiment, the present invention provides methods for extending the life span of a subject by administering an inhibitor of histone deacetylase to the subject in a suitable amount so as to extend the life of the subject. Extended life span can be determined by comparing the life spans of subjects of the same species receiving the inhibitor of histone deacetylase to the life span of controlled subjects (also of the same species) not receiving the inhibitor of histone deacetylase. The subjects that received the inhibitor of histone deacetylases exhibited extended life span compared to those subject that did not receive the inhibitor of histone deacetylase.

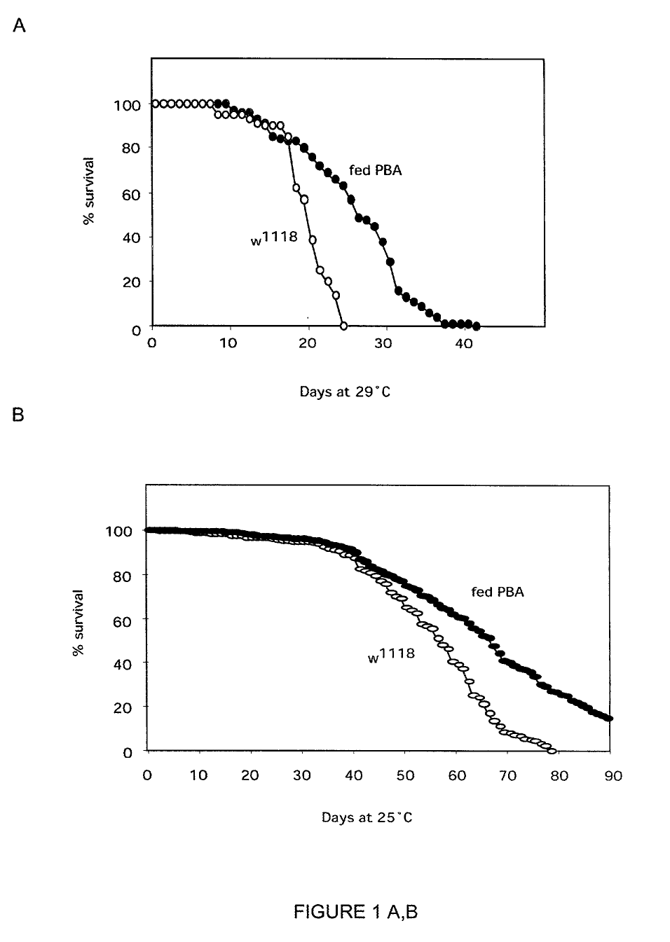

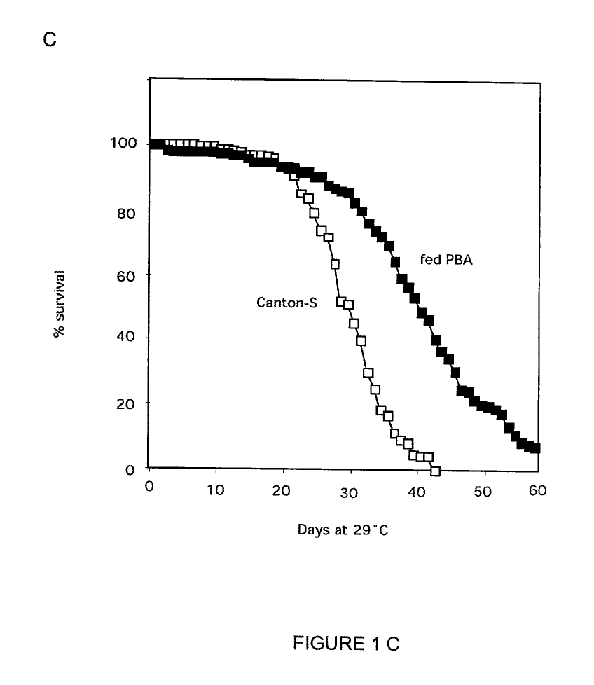

In a preferred embodiment, an inhibitor of histone deacetylase is the butyric acid derivative 4-phenyl butyrate (PBA). The PBA can be administered to a Drosophila (e.g., at room temperature) and the life span of the fly can be measured. Merely, as an example, this method is described in Example 1 infra. The PBA can extend the maximum life span of a Drosophila by e.g., 60%, while maintaining normal levels of activity and reproductive function (FIG. 1 and Table 1).

Butyric acid derivatives are examples of inhibitors of histone deacetylase. Examples of butyric acid derivatives include but, are not limited to isobutyramide, monobutyrin, tributyrin, 2-phenylbutyric acid, 3-phenylbutyric acid, 4-phenylbutyric acid (PBA), phenylacetic acid, cinnamic acid, alpha-methyldihydrocinnamic acid, 3-chloropropionic acid or vinyl acetic acid. The soluble form of the inhibitor of histone deacetylases or the salt of the butyric acid are preferred.

The subject of the invention can be a wild-type or a mutant subject. The subject of the invention can be a vertebrate or invertebrate organism. A preferred subject of the invention is a Drosophila melanogaster. The Drosophila strain w¹¹¹⁸ and Canton-S are especially preferred strains. In one embodiment of the invention, mutant Drosophila is used as the subject in the methods of the invention.

In addition, the present invention provides methods for identifying molecules that extends the life span of a subject. This method is carried out by administering to the subject a molecule and an inhibitor of histone deacetylase. Extension of life span can be determined by comparing the life span of the subject that received both the molecule and an inhibitor of histone deacetylase with the subject that only received the inhibitor of histone deacetylase but not the molecule of interest. The longer life span of the subject receiving both the molecule and the inhibitor of histone deacetylase identifies the molecule that further extends the life of the subject. In addition, the level of normal behavior of the subject can be assayed.

Examples of molecules of interest (or test molecules) include antioxidants (e.g. bioflavinoids, beta-carotenoids, vitamin C and E, Coenzyme Q, and free radical scavengers), cyclins (e.g. p53, P21 WAF1/Cip1, cyclin D1, bio 5495), anti-apoptotic factors (e.g. NAIP, Akt/PKB, CD23), hormones (e.g. somatotropin, IGF-1,hydroxy tryptophan) and growth factor (e.g. FGFs, TGFs, NGF, PDGF).

The present invention further provides methods for identifying molecular alterations in a subject administered with an inhibitor of histone deacetylase. Molecular alterations can be identified by comparing the presence, level or modification of nucleic acids or proteins in the subject that received the inhibitor of histone deacetylase with a subject that did not. Molecular alteration(s) in the subject that received the inhibitor of histone deacetylase identifies the molecular alteration effected by the molecule.

For example, a change in MRNA is indicative of a molecular alteration. Changes in mRNA include, but are not limited to, induction or repression of specific genes. In accordance with the practice of the invention, the changes in differential gene expression can be identified with a microarray membrane having e.g., Drosophila ESTs, and probed with mRNA isolated from e.g., Drosophila, treated with an inhibitor of histone deacetylase. Hybridization spots can be confirmed with e.g., RT-PCR.

Protein modification also can be indicative of a molecular alteration. Examples of protein modification in the subject administered with an inhibitor of histone deacetylase is the acetylation of histone, H3 and H4.

The induction of genes is another example of a molecular alteration. Genes that are induced in accord with the methods of the invention include, but are not limited to cytochrome P450, glutathione S-transferase 1-1, superoxide dismutase, transcription initiation factor TFIID 85kDa subunit, hepatocalcinoma-related transcription factor, daughterless protein, translation elongation factor 1 alpha, translation initiation factor 4 gamma, ribosomal protein L9, ribosomal protein L10A, ribosomal protein L21, ribosomal protein S8, ribosomal protein S9, ribosomal protein S12, ribosomal protein S15A, ribosomal , protein S24, ribosomal protein S29, ribosomal protein PO, ribosomal protein P2, hsc70, hsp60, dnaJ like2, angiotensin-converting enzyme-like protein, aminopeptidase, aminopeptidase N, serine protease, serine protease, serine proteinase 2, angiotensin-converting enzyme precursor, stubble, serine proteinase, cysteine proteinase 1, leucine aminopeptidase, trypsin theta precursor, growth factor-regulated tyrosine kinase substrate, guanine nucleotide-binding protein alpha, inactivation-no-afterpotential D, beta Adaptin (a), component of HA1 clathrin adaptor, guanyl-nucleotide exchange factor, epididymal secretory protein, SH2-SH3 adaptor protein, phosphorylase kinase gamma, p70-protein kinase(S6K), Fak like tyrosine kinase, Fps oncogene kinase, ADP/ATP translocase, mitochondrial phosphate carrier, sodium-dicarboxylate cotransporter, protein transport protein Sec23, neurotransmitter transporter, ADP/ATP translocase, transferrin precursor, putative odorant-binding protein A5 precursor, transportin, 26S proteasome subunit 4 ATPase, oxysterol-binding protein homolog Calphotin, T1/ST2 receptor binding protein precursor male specific protein, Neurocalcin homolog, ninaC, putative arginine-aspartate-rich RNA binding protein, TAR-binding protein, RNA binding protein, kurz protein, galactose-1-phosphate uridylyltransferase, mitochondrial aldehyde dehydrogenase, pyruvate kinase, aldehyde dehydrogenase 7, succinic semialdehyde dehydrogenase, citrate synthase, succinyl-CoA synthetase alpha subunit, dihydrolipoamide S-succinyltransferase, malate dehydrogenase, aspartate aminotransferase, serine-pyruvate aminotransferase 3-hydroxyisobutyrate dehydrogenase, 4-hydroxyphenylpyruvate dioxygenase, 4-amino butyrate amino transferase, haloacid dehalogenase-like hydrolase, phospholipase C, hydroxymethylglutaryl-CoA synthase alpha esterase, 1-acyl-glycerol-3-phosphate acyltransferase, fatty acid desaturase, amidophosphoribosylamidotransferase, ATP synthase subunit g, ATP synthase subunit, vacuolar ATP, synthase subunit, Rho small GTPase, hook, myosin heavy chain, p47 protein, metasis-associated1-like protein, protein involved in sexual development, Cdc37, cell division cycle 37 protein, X-linked nuclear protein, microsomal epoxide hydrolase, imaginal disc growth factor 1, 18s rRNA, and vitellogenin receptor.

Gene repression is also indicative of a molecular alteration. Genes that are repressed in accord with the methods of the invention may include, but are not limited to cystein proteinase 1, proteasome subunit, leucine aminopeptidase, mitochondrial processing protease-beta, ubiquitin conjugating enzyme, ribosomal protein S26, stubarista, ribosomal protein, dnaJ-1, guanine, nucleotide-binding protein gamma subunit, peroxisomal famesylated protein, midline fasciclin precursor, hexokinase, glyceraldehyde 3phosphate dehydrogenase 1, ATP synthase, subunit d, phosphogluconate, dehydrogenase, isocitrate dehydrogenase, aconitate hydratase precursor, acetyl-CoA carboxylase, hydroxyacyl-CoA dehydrogenase, AND-dependent 15-hydroxyprostaglandin dehydrogenase, fatty acid synthase, choline acetyltransferase, peptidyl gycine-alpha-hydroxylating monooxygenase, gamma-glutamylcysteine synthetase, tyrosine 3-monoxygenase, alpha-esterase, ATP synthase gamma, antennal-specific short-chain dehydrogenase/reductase, NADH:ubiquinone reductase 75kD subunit precursor, rhophilin, cytochrome c oxidase subunit Vib, cytochrome c oxidase, syntaxin, inorganic phosphate, cotransporter, tropomycine T, transferrin precursor, pheromone binding protein related protein 1 precursor, calreticulin, RNA helicase, osa, Cdk9, Mst87F, structural sperm protein, Transmembrane 4 Super Family, beta-spectrin, cut up, synaptogyrin homolog, tryptophanyl-tRNA synthetase, porin, and voltage dependent anion-selective channel.

In the present invention the identification of the presence, level and/or modification of proteins refers to changes that can be identified in the proteins of the subject that received an inhibitor of histone deacetylase. For example, proteins that can be modified include, but are not limited to, proteins involved in detoxification, transcription factors, proteins involved with translation, ribosomal proteins, chaperon proteins, peptidases, signal transduction, kinases, transporters, ligand binding or carriers, calcium binding proteins, calmodium biding proteins, RNA binding proteins, proteins involved with metabolism, structural proteins, membrane fusion proteins, proteins involved with metastasis, proteins involved with sexual development, cell cycle regulator proteins, nuclear proteins, microsomal enzymes, growth factors, proteins associated ribosomal RNA, protein receptors.

Another example of protein modifications that have been identified with the present invention is acetylation of histone H3 and H4. Other examples of protein modifications include, but are not limited to phosphorylation, methylation, and glycosylation.

The preferred agent used in the present invention to induce an extended life span in a subject is PBA. PBA inhibits the activity of histone deacetylase, thus inducing hyperacetylation of histones (Lea, M. A. & Randolph, V. M. Induction of reporter gene expression by inhibitors of histone deacetylase Anticancer Res. 18, 2717-2722 (1998)). This tends to release histones from their binding to chromatin, with consequent effects on gene transcription (i.e., increase expression or decrease expression). In addition, this invention shows that in Drosophila PBA induced life span extension results in a fly with normal behavior.

For example, the extension of life span of Drosophila by treatment with PBA serves as a useful model to identify genes involved in the ageing process. In Drosophila, transgenic constructs can be readily made to test for the effects of overexpression or silencing of individual genes. These transgenic flies can be used in the present invention to identify common features within control regions of different genes responsible for histone acetylation and regulatory regions within genes that control transcription. For example, overexpression of Sir2 protein, which has AND-dependent histone deacetylase activity, is implicated in silencing of gene transcription, and extends the budding life of yeast (Imai, S., Amstrong, C. M., Kaeberlein, M. & Guarente, L. The transcriptional silencing and longevity protein Sir2 is an AND-dependent histone deacetylase. Nature 403, 795-800 (2000)). Deletion of a histone deacetylase (RPD3) in yeast also extends life span (Kim, S., Benguria, A., Lai, C. & Jazwinski, S. M. Modulation of life-span by histone deacetylase genes in Saccharomyces cerrevisiae Mol. Biol. Cell 10, 3125-3136 (1999)) in yeast. These examples in yeast are consistent with the effect of PBA, a histone deacetylase inhibitor, in Drosophila.

The extension of life span of an organism may be a balance of expression of various genes that allow the organism to adjust to a changing physiological and cellular environment. Therefore, this invention provides a multicellular whole organism system that can identify these molecular changes associated with ageing or life span extension. And Drosophila represents a convenient model whole organism model for rapid identification of differential gene expression in a process such as ageing.

The compositions of the invention (inhibitors of histone deacetylase e.g., butyric acid derivatives and molecules of interest) can be administered using conventional modes of administration including, but not limited to, ingestion, absorption, intravenous (i.v.) administration, intraperitoneal (i.p.) administration, intramuscular (i.m.) administration, subcutaneous administration, oral administration, administration as a suppository, or as a topical contact, or the implantation of a slow-release device such as a miniosmotic pump, to the subject.

Also, the compositions may be in a variety of dosage forms, which include, but are not limited to, liquid solutions or suspensions, tablets, pills, powders, suppositories, polymeric microcapsules or microvesicles, liposomes, and injectable or infusible solutions. The preferred form depends upon the mode of administration.

The most effective mode of administration and dosage regimen for the compositions of this invention depends upon the subject's health and response to treatment. Accordingly, the dosages of the compositions should be titrated to the individual subject.

Dosage of the composition is dependant upon many factors including, but not limited to, the type of tissue affected, the type of subject, a subject's health, height, and weight, and a subject's response to the treatment with the compositions of the invention. Accordingly, dosages of the compositions of the invention can vary depending on the subject and the mode of administration. Administration of the compositions can be performed over various times. In addition, the administration can be repeated depending on factors as understood in the art.

The compositions also preferably include suitable carriers and adjuvants which include any material which when combined with the inhibitor of histone deacetylases and/or molecules of interest retains the molecule's activity and is non-reactive with the subject's immune system. Examples of suitable carriers and adjuvants include, but are not limited to, serum albumin; ion exchangers; alumina; lecithin; buffer substances, such as phosphates; glycine; sorbic acid; potassium sorbate; and salts or electrolytes, such as protamine sulfate. Other examples include any of the standard pharmaceutical carriers such as a phosphate buffered saline solution; water; emulsions, such as oil/water emulsion; and various types of wetting agents. Other carriers may also include sterile solutions; tablets, including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

The following examples are presented to illustrate the present invention and to assist one of ordinary skill in making and using the same. The examples are not intended in any way to otherwise limit the scope of the invention.

### EXAMPLE 1

### Extension of Life Span in Drosophila Fed PBA.

In this example data are provided on extension of life span in Drosophila flies by administering PBA. Flies are analyzed for normal function after being fed PBA.

(A) Drosophila Fed Various Concentrations of PBA

To test the action of PBA in vivo, newly eclosed adult flies (w¹¹⁸) are fed with standard fly medium (cornmeal, agar, dextrose, yeast) containing various concentrations of PBA (0, 0.1, 1, 2.5, 5, and 10 mM). The 4-phenylbutyric acid, sodium salt (PBA, Medicis, Scottsdale, Ariz.), was reported to be 99.6% pure. While the lower concentrations of PBAhad no effect on longevity, flies fed with medium containing 10 mM PBA showed about 35% extension in median life span, and 60% extension in maximum life span (FIG. 1A). Life span is measured at 29° C. (FIG. 1A) and at 25° C. (FIG. 1B). FIG. 1C shows the Canton-S strain of Drosophila fed 10 mM PBA has an extended life span as compared to the same strain fed on fly medium.

In another embodiment of the invention other known inhibitors of histone deacetylase (HDAC) will be used to extend the life span of the organism, for example Trichostatin A (TSA), trapoxin, sodium butyrate, and suberoylanilide hydroxamic acid (SAHA). Binding of these inhibitors to HDAC inhibits its enzyme activity, which induces hyperacetylation of histones that affects gene expression. These molecular alterations will be analyzed as described in Example 2, infra.

(B) Drosophila Fed PBA at Various Time Points

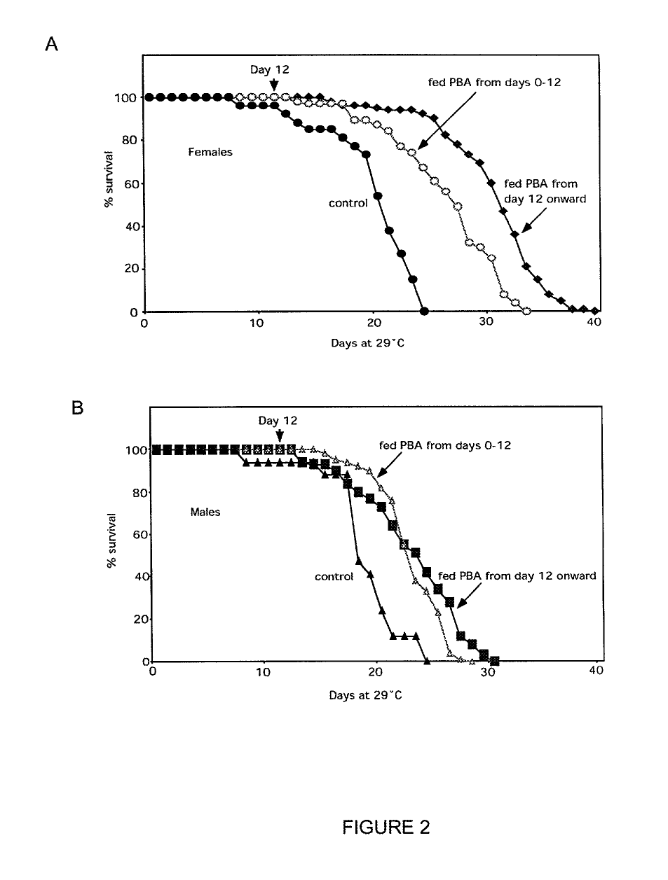

It has been suggested that early events in life can delay the onset of ageing, thus extending longevity (Arking, R. Biology of Aging: observations and principles (Sinauer, ed. 2 Sunderland, 1998). However, life span extension by PBA occurs whether it is fed early or late in life (FIG. 2). Newly emerged adult flies were fed with PBA from emergence to 12 days (before survival ordinarily begins its rapid decline), then, transferred to plain medium for the rest of their lifetime. Alternatively, flies fed plain medium for their first 12 days, then medium containing the drug for the remainder of life. In both cases, PBA-treated flies showed increased life span compared with untreated flies (FIG. 2). In virgin female flies (but not in males) treatment initiated at a later age was even more effective than the same duration of treatment at young age (FIG. 2A). The results indeed show that establishment of an altered cellular environment by PBA, albeit for a limited period of time, does extend the life span of flies. One explanation for these results is that PBA inhibits the accumulation of cellular and molecular damage, and/or stimulates cellular repair mechanisms.

In another embodiment of the invention aged flies can be used treated with PBA to identify molecular alterations in very old flies. After twelve days at 29° C. the survival of flies decline very rapidly. Flies from emergence to 16 days, 20 days, and 22 days will be collected and fed with PBA for the remainder of life and, then their life span will be monitored and molecular alterations analyzed.

In another embodiment of the invention PBA is fed to flies for limited periods of time. It will also be very interesting to determine the minimum period of PBA treatment required for extension of life span. To make this determination, we will treat newly emerged adult flies with PBA. Each group of flies will be fed medium containing 10 mM PBA for 1 to 12 days, then transferred to plain medium for the remainder of life. The survival of those flies in each group will be measured. After obtaining this result, flies from different age groups will be treated with PBA for the minimum period of time to determine whether such duration is still effective for different age groups.

(C) The Role of Caloric Restriction in PBA Fed Drosophila

Caloric restriction increases life span in rodents, worms, and yeast (Guarente, L & Kenyon, C. Nature 408, 255-262 (2000)). Although PBA is odorless and tasteless to human, flies may dislike the smell or taste of the drug, which may cause caloric restriction. To test the issue of possible caloric restriction effects, the intake of food was measured by adding food dye to both the control and PBA containing media. After an overnight feeding, the alimentary tracts of ten flies from each group were dissected out and examined for color. No difference in the alimentary tract of these flies was discernible. Further, the weight and size of the flies were measured after ten days of feeding with or without PBA (Table 1). Again, no differences were observed. Therefore, the extension of life span observed in Drosophila fed PBA is most likely not due to caloric restriction.

(D) Reproduction in Drosophila Fed PBA

Reproductive signaling in C. elegans and Drosophila also contribute to extend life span (Guarente, L & Kenyon, C. Nature 408, 255-262 (2000)). PBA treatment may slow down reproduction of flies and thus results in the extension of life span. The role of PBA on the reproductive activity of female Drosophila was examined by counting the number of eggs layed, the percentage of eggs yielding adult progeny, and the weight and size of the progeny. In all the measurements showed no detrimental effect of feeding PBA to the reproductive activity of the female flies (Table 1). The suggests that PBA does not effect fly reproduction and can be used on a long term to extend the life span of wild-type and mutant flies.

TABLE 1
Weight, Size and Reproductive ability of Drosophila Fed PBA

| | |
|---|---|
| control | PBA |

A. Parent

| | | |
|---|---|---|
| weight of 5 flies (mg) (n = 10) | | |
| females | 5.1 ± 0.2 | 5.1 ± 0.2 |
| males | 3.3 ± 0.1 | 3.3 ± 0.4 |
| size of fly (mm) (n = 40) | | |
| female | 5.5 ± 0.1 | 5.5 ± 0.1 |
| male | 4.5 ± 0.1 | 4.6 ± 0.1 |
| egg laying (3 males + 3 females a vial for 16 hrs) | 66 ± 15 | 70 ± 16 |
| (n = 20 vials) | | |
| % of eggs yielding adults (%) (n = 20 vials) | 66 ± 15 | 70 ± 16 |

B. Progeny

| | | |
|---|---|---|
| weight of 5 flies (mg) (n = 10) | | |
| females | 5.3 ± 0.4 | 5.2 ± 0.2 |
| males | 3.4 ± 0.6 | 3.5 ± 0.3 |
| size of fly (mm) (n = 40) | | |
| female | 5.4 ± 0.2 | 5.5 ± 0.1 |
| male | 4.5 ± 0.2 | 4.6 ± 0.2 |

(E) Activity Level of Drosophila fed PBA

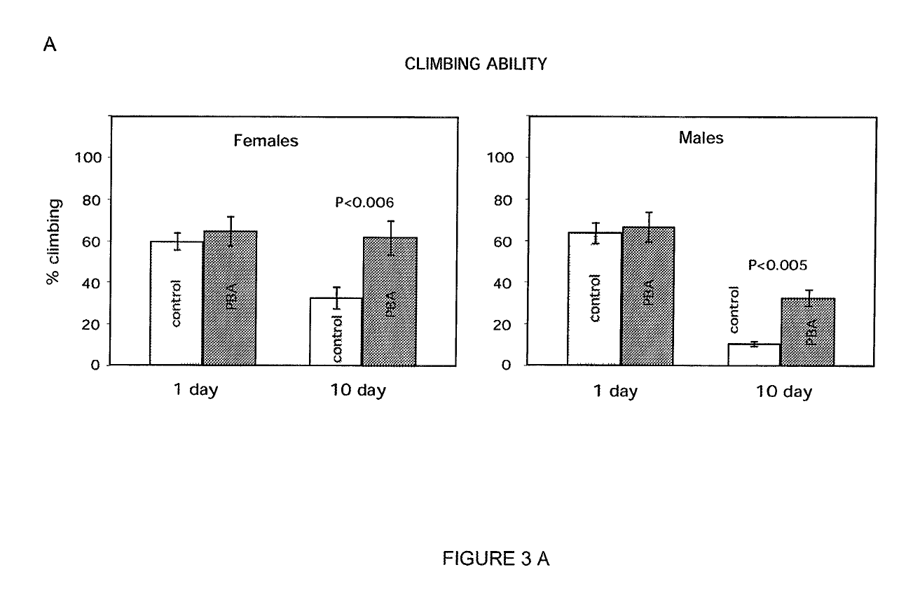

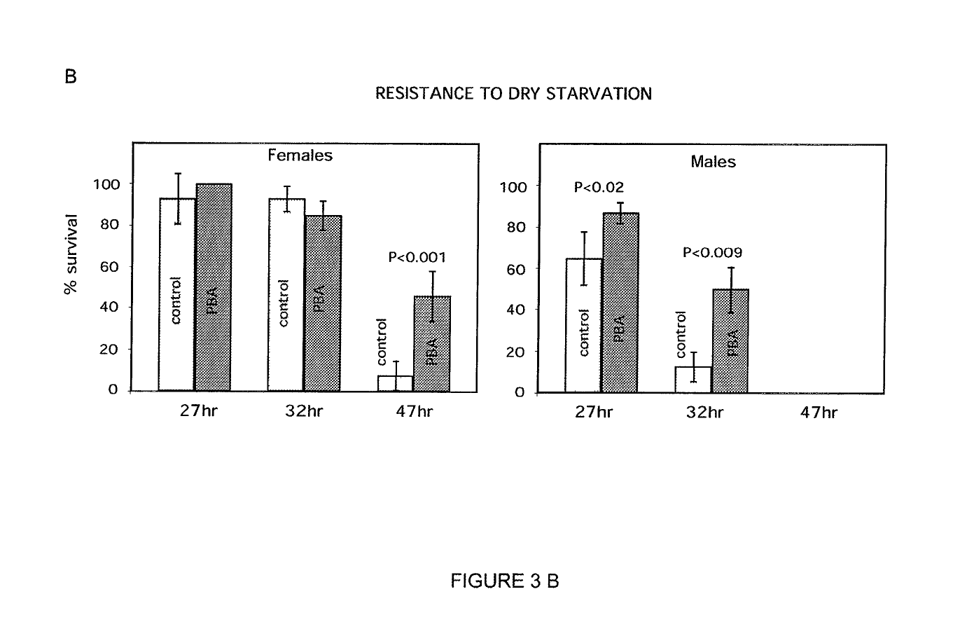

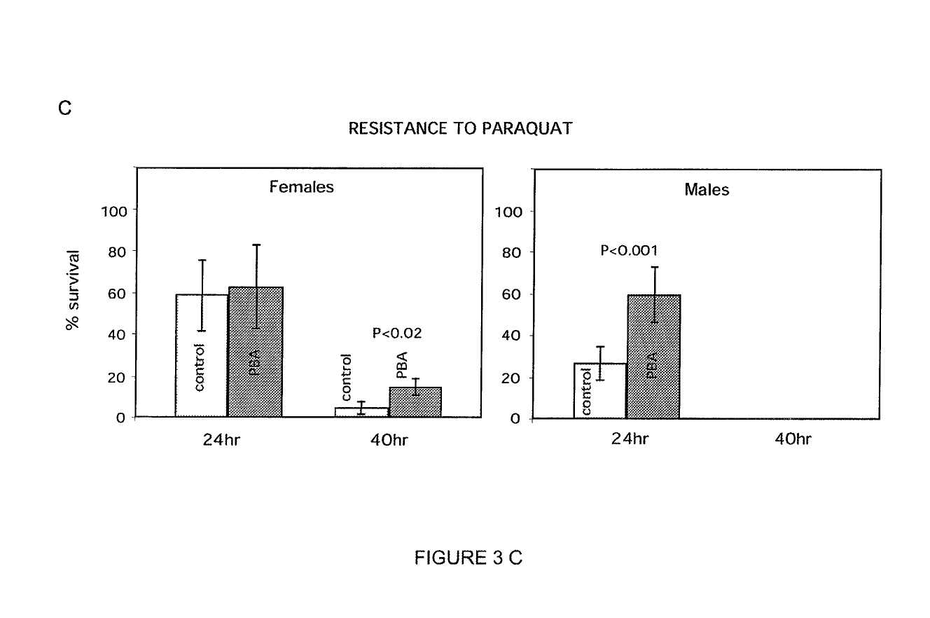

Life extension without maintaining physical or mental vigor is undesirable. To examine whether the life span increase by PBA is associated with extended maintenance of vigor, the locomotor activity was measured for young and old flies raised with and without PBA treatment. A known assay of negative geotaxis in a counter current distribution apparatus was measured the locomotor activity of the flies (Benzer, S. Proc. Natl. Acad. Sci. U.S.A. 58, 1112-1119 (1967); FIG. 3A). The results showed that PBA enhanced the climbing ability of old flies significantly.

(F) Resistance to Stress in Drosophila Fed PBA

Stress to an animal can be achieved by starvation or an increase production of free radicals. The effective removal of free radicals or the increase of antioxidant levels reduce stress to the animal and are important in slowing down the ageing process. The failure to repair oxidative damages and the elevation of free radial is known to shorten the life span of animal. Therefore, resistance to stress was measured in PBA fed flies by measuring if they have an increase resistance to the free radical generator, paraquat, and to dry starvation. PBA treated and control flies drank sucrose solution containing 20 mM paraquat were tested for duration of life span (e.g. survival) (FIG. 3C). PBA fed flies survived much longer under paraquat induced stressed conditions than control flies did. PBA treated and control flies were also put into empty vials to measure their survival on dry starvation (FIG. 3B). PBA treatment of Drosophila enhanced the resistance to starvation.

### EXAMPLE 2

### Molecular Alterations in Drosophila Fed PBA.

(A) Levels of Histone Acetylation in Drosophila Fed PBA.

PBA, an inhibitor of histone deacetylase, is known to enhance acetylation in the tails of histones H3 and H4, which causes the tails to be released from the DNA (Lea et al. Induction of Histone Acetylation and Growth Regulation in Erythroleukemia Cells by 4-phenylbutyrate and Structural Analogs Anticancer Research 19:1971-1976 (1999)). The acetylation of histones changes the binding affinity between histones and DNA, and leads genes to recruit transcription factors and undergo transcription. Using western blots stained with specific antibodies, the level of histone acetylation was measured in Drosophila, with and without PBA treatment. Batches of 100 flies (males+females) were used to prepare histones (Alfageme, C. R., Zweidler, A., Mahowald, A. & Cohen, L. H. Histones of Drosophila embryos J. Biol. Chem. 12, 3729-3736 (1974)). The homogenates of whole flies were centrifuged at 2,500×g for 10 min in a medium containing 0.05 M glycine, 10 mM Tris potassium maleate, 5 mM MgCl₂, 10 mM mercaptoethanol, pH 7.3. Isolated nuclei were used to extract histones by HCl treatment. 10 μg samples of histone protein were loaded on a 16.5% polyacrylamide gel for electrophoresis, then transferred to a PVDF membrane (Immobilin-P transfer membrane, Millipore, Bedford, Mass.), followed by hybridization with antibodies to acetylated and nonacetylated H3 and H4 (Upstate Biotechnology, Lake Placid, N.Y.).

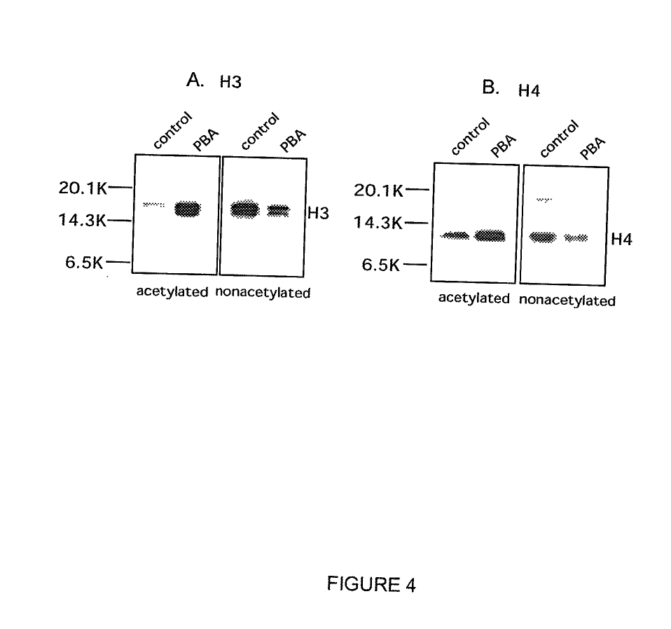

FIG. 4A and 4B, show that the nonacetylated forms of the histone proteins prevailed in untreated flies, while flies fed PBA had an increase of acetylated H3 and H4. This global change in histone acetylation suggests that the modification of chromatin structure may change the regulation of transcription.

(B) Differential Gene Expression in Drosophilas Fed PBA:

To investigate the differential pattern of gene activity resulting from PBA treatment, high density membrane arrays containing about 27,000 Drosophila EST clones (Research Genetics, Huntsville, Tenn.) were used. Messenger RNA was prepared from either 10 day old flies that had been treated with 10 mM PBA at 29° C. from emergence onward. A similar preparation was made from untreated flies. These RNA preparations were used as probes on the membrane arrays.

The Smart PCR cDNA synthesis kit (Clontech, Palo Alto, Calif.) was used to synthesize probes for hybridization. Total RNA was prepared from flies (males+females) fed for 10 days at 29° C. with medium containing 10 mM PBA or plain medium. After first strand synthesis of cDNAs by MMLV reverse transcriptase (M-MLV reverse transcriptase, Boehringer Mannheim, Indianapolis, Ind.), the cDNA was amplified by polymerase chain reaction (PCR). The reaction consisted of 95° C. for 1 minute, then 24 cycles of 95° C. for 15 seconds, 65° C. for 30 seconds, and 68° C. for 6 minutes. Probes were prepared by a random primed DNA labeling method with p³². Filters containing 27,000 Drosophila EST clones (Research Genetics, Huntsville, Tenn.) were prehybridized for 4 hours, then probes were added to hybridize for 16 hours at 58° C. in a buffer containing 1 M NaCl, 0.05M Tris (pH 8.0), 5 mM EDTA, 1% SDS, and 10% Dextran Sulfate. After hybridization, filters were washed several times and exposed to X-ray film.

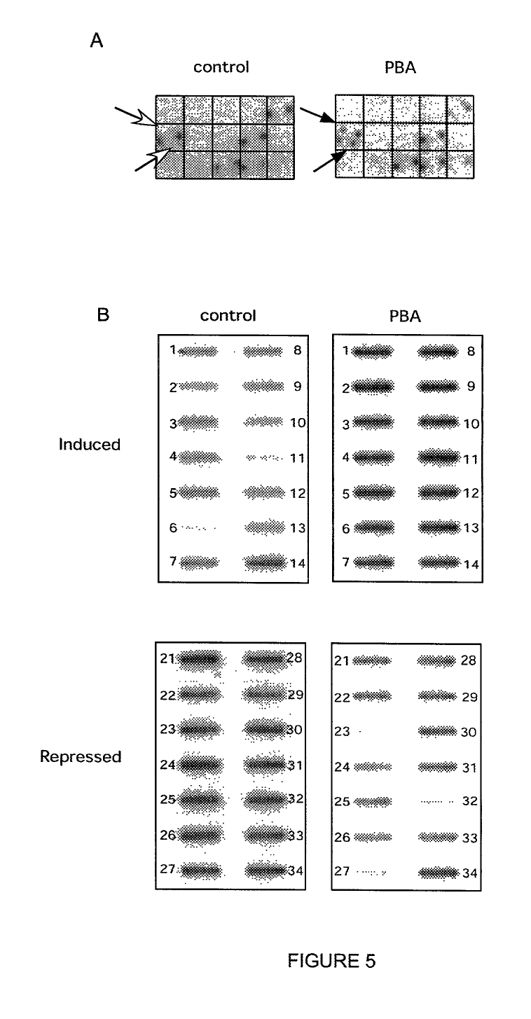

In FIG. 5A a sample portion of the membrane array hybridized with each of the two different probes described above is shown. A stringent criterion was used to distinguish differences in hybridization spot intensity. One hundred genes that were strongly induced by PBA were identified, and 48 genes that were suppressed by the same treatment were identified, as judged by the disappearance of a visible spot. Due to the strict criteria used to identify these genes, one can predict that PBA treatment induced a large change in the expression level of these genes. This pattern of gene expression was reproducible. These result show that PBA can create a cellular environment that leads to an altered pattern of gene expression and altered protein modification. These molecular alterations are likely to be responsible for the extended life span observed in PBA treated flies.

Reverse northern blot analysis confirmed a subset of genes that were identified in the microarray (FIG. 5B). Among the 100 induced genes, 3 are involved in detoxification, 3 are chaperon proteins, 2 are involved in the translation machinery, 3 are transcription factors, 7 are involved in signal transduction pathways, 25 are involved in metabolism, 11 are ribosomal proteins, 11 are proteases, 4 are kinases, 13 function as transporters or carriers, and 18 are involved in other miscellaneous functions. Among the 48 repressed genes, 21 are involved in metabolism, 4 are proteases, 2 are ribosomal proteins, and 21 others have miscellaneous functions (Table 2).

TABLE 2
Genes that are Induced or Repressed in Drosophila Fed PBA

Genes induced in PBA treated flies

| function | clone ID | Gene |
|---|---|---|
| detoxification | GH18513 | cytochrome P450 |
| | GH16867 | glutathione S-transferase 1—1 |
| | GH02759 | superoxide dismutase |
| transcription factor | GH19265 | transcription initiation factor TFIID 85kDa subunit |
| | GH13534 | hepatocalcinoma-related transcription factor |
| | GH10651 | daughterless protein |
| translation | GH24069 | translation elongation factor 1alpha |
| | GH04045 | translation initiation factor 4 gamma |
| ribosomal protein | GH03579 | ribosomal protein L9 |
| | GH05501 | ribosomal protein L10A |
| | GH17295 | ribosomal protein L21 |
| | GH04990 | ribosomal protein S8 |
| | GH22258 | ribosomal protein S9 |
| | GH05877 | ribosomal protein S12 |
| | GH04971 | ribosomal protein S15A |
| | GH12633 | ribosomal protein S24 |
| | GH22282 | ribosomal protein S29 |
| | GH06043 | ribosomal protein P0 |
| | GH27908 | ribosomal protein P2 |
| chaperon | GH03156 | hsc70 |
| | GH15852 | hsp60 |
| | GH23459 | dnaJ like2 |
| peptidase | GH10096 | angiotensine-converting enzyme-like protein |
| | GH19035 | Aminopeptidase |
| | GH02922 | aminopeptidase N |
| | GH27878 | serine protease |
| | GH17983 | serine protease |
| | GH27528 | serine proteinase 2 |
| | GH27268 | angiotensine-converting enzyme precursor |
| | GH24919 | stubble, serine proteinase |
| | GH11427 | cysteine proteinase 1 |
| | GH18236 | leucine aminopeptidase |
| | GH08068 | trypsin theta precursor |
| signal transduction | GH12653 | growth factor-regulated tyrosine kinase substrate |
| | GH08039 | guanine nucleotide-binding protein alpha |
| | GH11552 | inactivation-no-afterpotential D |
| | GH24463 | beta Adaptin (a component of HA1 clathrin adaptor) |
| | GH19850 | guanyl-nucleotide exchange factor |
| | GH22047 | epididymal secretory protein |
| | GH27474 | SH2-SH3 adaptor protein |
| kinase | GH20420 | phosphorylase kinase gamma |
| | GH02870 | p70-protein kinase(S6K) |
| | GH02782 | Fak like tyrosine kinase |
| | GH20864 | Fps oncogene kinase |
| transporter | GH21002 | ADP/ATP translocase |
| | GH16061 | Mitochondrial phosphate carrier |
| | GH25396 | sodium-dicarboxylate cotransporter |
| | GH10571 | protein transport protein Sec23 |
| | GH25034 | neurotransmitter transporter |
| | GH24374 | ADP/ATP translocase |
| ligand binding or carrier | GH02726 | transferrin precursor |
| | GH25425 | putative odorant-binding protein A5 precursor |
| | GH07364 | transportin |
| | GH05348 | 26S proteasome subunit 4 ATPase |
| | GH12064 | oxysterol-binding protein homolog |
| | GH21620 | Calphotin |
| | GH21271 | T1/ST2 receptor binding protein precursor |
| | GH19150 | male specific protein |
| Calcium binding | GH15907 | Neurocalcin homolog |
| calmodium biding | GH16421 | ninaC |
| RNA binding | GH06521 | putative arginine-aspartate-rich RNA binding protein |
| | GH14113 | TAR-binding protein |
| | GH23688 | RNA binding protein |
| | GH04462 | kurz protein |
| metabolism | GH23685 | galactose-1-phosphate uridylyltransferase |
| | GH11111 | mitochondrial aldehyde dehydrogenase |
| | GH14417 | pyruvate kinase |
| | GH25529 | aldehyde dehydrogenase 7 |
| | GH19428 | succinic semialdehyde dehydrogenase |
| | GH23763 | citrate synthase |
| | GH11074 | succinyl-CoA synthetase alpha subunit |
| | GH02169 | dihydrolipoamide S-succinyltransferase |
| | GH15791 | malate dehydrogenase |
| | GH02970 | aspartate aminotransferase |
| | GH27315 | serine-pyruvate aminotransferase |
| | GH06781 | 3-hydroxyisobutyrate dehydrogenase |
| | GH11957 | 4-hydroxyphenylpyruvate dioxygenase |
| | GH04328 | 4-amino butyrate amino transferase |
| | GH03365 | haloacid dehalogenase-like hydrolase |
| | GH24632 | phospholipase C |
| | GH10359 | hydroxymethylglutaryl-CoA synthase |
| | GH12017 | alpha esterase |
| | GH22114 | 1-acyl-glycerol-3-phosphate acyltransferase |
| | GH27450 | Fatty acid desaturase |
| | GH11340 | amidophosphoribosylamidotransferase |
| | GH15786 | ATP synthase subunit g |
| | GH07365 | ATP synthase subunit |
| | GH21154 | vacuolar ATP synthase subunit |
| | GH15180 | Rho small GTPase |
| structural protein | GH24204 | hook |
| | GH13052 | myosin heavy chain |
| membrane fusion | GH25555 | p47 protein |
| metastasis | GH27854 | metasis-associated 1-like protein |
| sexual development | GH11622 | protein involved in sexual development |
| cell cycle regulator | GH20989 | Cdc37, cell division cycle 37 protein |
| Nuclear protein | GH16039 | X-linked nuclear protein |
| microsomal enzyme | GH27556 | microsomal epoxide hydrolase |
| Growth factor | GH04843 | imaginal disc growth factor 1 |
| ribosomal RNA | GH23808 | 18s rRNA |
| receptor | GH21958 | vitellogenin receptor |

Genes repressed in PBA treated flies

| function | clone ID | gene |
|---|---|---|
| peptidase | GH19855 | cystein proteinase 1 |
| | GH01155 | proteasome subunit |
| | GH04837 | leucine aminopeptidase |
| | GH24325 | mitochondrial processing protease-beta |
| | GH19904 | ubiquitin conjugating enzyme |
| ribosomal protein | GH13719 | ribosomal protein S26 |
| | GH20634 | stubarista, ribosomal protein |
| chaperonin | GH08162 | dnaJ - 1 |
| signal transduction | GH21285 | guanine nucleotide-binding protein gamma subunit |
| | GH03076 | peroxisomal farnesylated protein |
| | GH25149 | midline fasciclin precursor |
| metabolism | GH07287 | hexokinase |
| | GH04145 | glyceraldehyde 3phosphate dehydrogenase 1 |
| | GH03431 | ATP synthase, subunit d |
| | GH21857 | phosphogluconate dehydrogenase |
| | GH08961 | isocitrate dehydrogenase |
| | GH20491 | aconitate hydratase precursor |
| | GH27434 | acetyl-CoA carboxylase |
| | GH24961 | hydroxyacyl-CoA dehydrogenase |
| | GH09745 | NAD-dependent 15-hydroxyprostaglandin dehydrogenase |
| | GH20967 | fatty acid synthase |
| | GH25173 | choline acetyltransferase |
| | GH09669 | peptidyl gycine-alpha-hydroxylating monooxygenase |
| | GH03051 | gamma-glutamylcysteine synthetase |
| | GH02304 | tyrosine 3-monoxygenase |
| | GH11805 | alpha-esterase |
| | GH08466 | ATP synthase gamma |
| | GH15584 | antennal-specific short-chain dehydrogenase/reductase |
| | GH22254 | NADH:ubiquinone reductase 75kD subunit precursor |
| | GH07977 | rhophilin |
| | GH09403 | cytochrome c oxidase subunit Vib |
| | GH24996 | cytochrome c oxidase |
| transporter | GH04326 | syntaxin |
| | GH27743 | inorganic phosphate cotransporter |
| ligand binding or carrier | GH03102 | tropomycine T |
| | GH19291 | transferrin precursor |
| | GH26863 | pheromone binding protein related protein 1 precursor |
| | GH25160 | calreticulin |
| RNA binding | GH24068 | RNA helicase |
| DNA binding | GH03026 | osa |
| kinase | GH21935 | Cdk9 |
| structural protein | GH23081 | Mst87F, structural sperm protein |
| | GH05525 | Transmembrane 4 Super Family |
| | GH22816 | beta-spectrin |
| motor protein | GH08464 | cut up |
| Synaptic plasticity | GH22872 | synaptogyrin homolog |
| synthetase | GH26676 | tryptophanyl-tRNA synthetase |
| ion channel | GH08586 | porin, voltage dependent anion-selective channel |

In another embodiment of the invention RT-PCR was be used to verify the changes in transcription levels seen by the difference in hybridization on the membrane arrays. Newly emerged adult flies (males+females) were fed food containing 10 mM PBA, or plain food for 10 days at 29° C., then total RNA was extracted and used to synthesize cDNA. For each PCR reaction, 50 ng of cDNA was used. Primers were designed from the sequence of each gene to detect fragments of between 300 and 600 base pairs (Table 4). PCR reactions were performed with 30 cycles of 94° C. for 1 minute, 55° C. for 1 minute, and 72° C. for 1 minute.

TABLE 4

Primer Sequences Used For RT-PCR

| | | | |
|---|---|---|---|
| gene name | gene ID | oligo ID | oligo sequence |

| | | | |
|---|---|---|---|
| superoxide dismutase | CG8905 | GH191 | GCTGGTACCAATTTATTAGCCGCAAC |
| | | GH192 | TGATCTGAAGAAGGCCATCGAGTCGC |
| cytochrome P450-4d1 | CG3656 | GH171 | CGAAATGTGGCTCCTACTATCGCTAGT |
| | | GH172 | ACTTGCGTCCGTTGCTCACCAGCAGT |
| glutathion S-transferase | CG10045 | GH231 | CAGTGTACATCGCGAGTTTCACAAC |
| | | GH232 | TCCAGGAAGGTGTTCAGGAACTCGAA |
| hsc70 | CG4264 | GH011 | CCAGTTTGATCGAAGGTGCGGCAGA |
| | | GH012 | TGTCCAGACCGTAAGCGATAGCAGCG |
| hsp60 | CG12101 | GH101 | AGGCAAATATCAGTCAACATGATGCGCA |
| | | GH102 | CCTTGACCGTCTCGATGGCTAGCAT |
| dnaJ-like2 | CG3061 | GR151 | GGAGAGGCTCTTTCCTACGGATAATGCC |
| | | GH152 | ATATCCCATACTCGTTGTTGTAGTATTGCC |
| elongation factor 1alpha | CG8280 | GH061 | ACATTGTCGTGATCGGACACGTCGA |
| | | GH062 | TATGGTGGCTCGGAGGAGTCCATCTTGTT |
| Inebriated | CG15444 | GH141 | CTTGAGGCACAGCCAACTCTCTGATAG |
| | | GH142 | TAACCGCGACTTCAGCTCCATGCTGA |
| daughterless, specific RNA | CG5102 | GH051 | GCCAGTTTGAAACTCGATCGCAGTGC |
| polymerase II transcription factor | | | |
| | | GH052 | CGGTATCATGTGATGCTGGGCACTTA |
| Transportin | CG7398 | GH181 | AAAGCACAGCCAACACCCAAAGCAGCAAA |
| | | GH182 | GGCACTCGTGTTTGATATACTCCACGATC |
| epdidymal secretory protein | CG7291 | GH201 | TAGATTCGTAGCGCTGTGAAGAGGCA |
| | | GH202 | AAAATCAGGAGTGCTCAGTGCGCTCTC |
| mitochondrial phosphate carrier | CG4994 | GH111 | TAACGTTGCTGACGAATACCGACCC |
| protein | | | |
| | | GH112 | AGATACAAGGAGGTGCGGTACAGGTA |
| imaginal disc growth factor 1 | CG4472 | GH161 | TTTGGCCAGTGCAAAGTCCACGGAAGT |
| | | GH162 | AGCTCCGATTTTCTTCCAGGACGAAC |
| glyceraldehyde 3phosphate | CG12055 | GH241 | GCTCTGCATATACTTGATCAGGTCGATG |
| dehydrogenase 1 | | | |
| | | GH242 | AATGTCTCCGTTGTGGATCTTACCG |
| NADA:ubiquinone reductase | CG2286 | GH261 | TGAGAACGAGGACGTCAACGAGGAATG |
| 75kD subunit precursor | | | |
| | | GH262 | TGCAGTTCGTTGTGCACATAGGCCTTG |
| cytochrome c oxidase | FBgn0013674 | GH271 | GGACATCCTGGAGCATTAATTGGAGATG |
| | | GH272 | TCCAGCGGATAGAGGTGGATAAACAG |
| peptidyl gycine-alpha- | CG3832 | GH331 | GTTCGAATACGGTGAAAATGCCACGC |
| hydroxylating monooxygenase | | | |
| | | GH332 | AGTTCTTGCCCACCTTGAAACCCACT |
| fatty acid synthase | CG3523 | GH381 | ATTTGTGAGAGCGGTAGCTTGGCGGTTTC |
| | | GH382 | AGTCTCAACCTGTTCCTCCTTGGTGAGG |
| cytochrome c oxidase subunit Vib | CG14235 | GH321 | ATCGTCAGACAGCAGCAACATGTCCGCCTA |
| | | GH322 | TCTGCATTCACCGCTGGGGAAAGCACA |
| Hexokinase | CG3001 | GH301 | CTCACAAATGCCCTACGTATGCACAT |
| | | GH302 | AAGTAGGATGGATAGGGAGCTGGAGCT |
| DnaJ like 1 | CG10578 | GH311 | TTCTTTGGATCGTCGGATCCGTTTGG |
| | | GH312 | CTCGTGGTTCGGATTCACCTGTATCCT |
| Osa | CG7467 | GH291 | CGATGACTCAACAGTCCAGTTCTTTGGC |
| | | GH292 | TTAGGCTGTACTCGCACTTGACCCAAA |
| Calrecticulin | CG9429 | GH391 | AGTTCGGACAACCATCGGAGTTGGAAG |
| | | GH392 | AGCAGTCGAACAGCTTCACATAGCCG |
| peroxisomal farnesylated protein | CG5325 | GH281 | AAACGACTTGCTGGACAGTGCTCTCCA |
| | | GH282 | TGGTAGGAACATGTTTCCATCACCCTC |
| cycline-dependent kinase9 | CG5179 | GH451 | TGTCGGCTTCTCGCGAAACTGTGATTGT |
| | | GH452 | CTTGACGTTCATGTTGGACAGAAGACC |
| Porin | CG6647 | GH251 | ATACAGCGATTTGGGCAAACAGGCTCG |
| | | GH252 | CCATCGTTGACAGCTGTGTGCAGAACAA |
| Opsin | CG4550 | opsin1 | CGATACTTTCCTCTGTACATTGCAGAC |
| | | opsin2 | TGCTAACCAGAACATCCAGTGGATCC |

Seven of the induced genes, previously shown to be involved in longevity, include superoxide dismutase, elongation factor1-alpha, glutathione S transferase, cytochrome P450, and three chaperon proteins. In Drosophila, transgenic flies with multiple copies of superoxide dismutase genes have been reported to show extended mean life span (Orr, W. C. & Sohal, R. S. Extension of life-span by overexpression of superoxide dismutase and catalase in Drosophila melanogaster. Science 263, 1128-1130 (1994); Parkes, T. L. et al. Extension of Drosophila lifespan by overexpression of human SOD1 in motoneurons. Nat. Genet. 19, 171-174 (1998); Orr, W. C. & Sohal, R. C. Effects of Cu/Zn superoxide dismutase overexpresion on life span and resistance to oxidative stress in transgenic Drosophila melanogaster. Arch. Biochem. Biophys. 301, 34-40 (1993)). Elongation factor1-alpha plays a critical role in maintaining the level of protein synthesis, which normally declines with age (Webster, G. C. & Webster, S. L. Specific disappearance of translatable messenger RNA for elongation factor one in aging Drosophila melanogaster. Mech. Ageing Devel. 24, 335-342). Glutathione S transferase and cytochrome P450 are involved in detoxification, one of the determinants of ageing (Arking, R. Biology of Aging: observations and principles (Sinauer, ed. 2 Sunderland, 1998); Mannervik, B. The isoenzymes of glutathione transferase. Adv. Enzymol. Relat. Areas Mol. Biol. 57, 357-417 (1985)). Heat shock proteins enhance resistance to stress and extend life span (Tatar, M., Khazaeli, A. A. & Curtsinger, J. W. Chaperoning extended life. Nature 390, 30 (1997); Lithgow, G. J. Temperature, stress response and aging. Rev. Clin. Gerontol. 6, 119-127 (1996)). All of these genes are induced by PBA, as confirmed in FIG. 5. In addition, 19 other genes, six from the induced group, and 13 from the repressed group; all confirmed by RT-PCR, support the membrane array screening method used to identify these genes is accurate. These 19 genes are listed in Table 3 and grouped according to their putative functions. Table 2 is a complete list of the 100 induced and 48 repressed genes discussed above.

TABLE 3
Genes Induced or Repressed in PBA Fed Drosophila

GENES INDUCED IN PBA-TREATED FLIES (confirmed by Northern)

| Function | Clone ID | Gene | Fold-change |
|---|---|---|---|
| detoxification | CG8905 | superoxide dismutase | 51.9 |
| | CG3656 | cytochrome P450-4d1 | 7.2 |
| | CG10045 | glutathione S transferase | 4.6 |
| chaperon | CG4264 | hsc70 | 4.5 |
| | CG12101 | hsp60 | 6.7 |
| | CG3061 | dnaJ like2 | 2.9 |
| translation | CG8280 | translational elongation factor1 α | 4.1 |
| neurotransmitter | CG15444 | inebriated | 26.8 |
| transcription factor | CG5102 | daughterless | 8.0 |
| ligand binding | CG7398 | transportin | 8.0 |
| signal transduction | CG7291 | epididymal secretory protein | 7.5 |
| transporter | CG4994 | mitochondrial phosphate carrier protein | 5.1 |
| growth factor | CG4472 | imaginal disc growth factor1 | 5.3 |

GENES REPRESSED IN PBA-TREATED FLIES (confirmed by Northern)

| | | | |
|---|---|---|---|
| | | | Fold- |
| Function | Clone ID | Gene | Change |
| metabolism | CG12055 | glyceraldehyde 3 phosphate dehydrogenase1 | 3.7 |
| | CG2286 | NADH:ubiquinone reductase 75kD subunit | 25.3 |
| | | precursor | |
| | FBgn0013674 | cytochrome c oxidase | 6.6 |
| | CG3832 | peptidyl glycine α hydroxylating | 1.4 |
| | | monooxygenase | |
| | CG3523 | fatty acid synthetase | 2.4 |
| | CG14235 | cytochrome c oxidase subunit Vlb | 2.2 |
| | CG3001 | hexokinase | 5.0 |
| chaperonin | CG10578 | dnaJ like1 | 13.2 |
| DNA binding | GH03026 | osa | 5.0 |
| ligand binding | GH25160 | calreticulin | 26.5 |
| signal transduction | GH03076 | peroxisomal farnesylated protein | 1.8 |
| kinase | GH21935 | cyclin-dependent kinase9 | 2.7 |
| ion channel | GH08586 | porin | 1.4 |

What is claimed:

1. A method for extending the life span of a subject comprising administering an inhibitor of histone deacetylase to the subject in an amount effective to extend the life span.

2. The method of claim 1, wherein the inhibitor of histone deacetylase is a butyric acid derivative.

3. The method of claim 2, wherein the butyric acid derivative is selected from the group consisting of isobutyramide, monobutyrin, tributyrin, 2-phenylbutyric acid, 3-phenylbutyric acid, 4-phenylbutyric acid (PBA), phenylacetic acid, cinnamic acid, alpha-methyldihydrocinnamic acid, 3-chloropropionic acid and vinyl acetic acid.

4. The method of claim 3, wherein the butyric acid derivative is soluble.

5. The method of claim 3, wherein the butyric acid derivative is a salt.

6. The method of claim 1, wherein the inhibitor of histone deacetylase is PBA and PBA is a salt.

7. The method of claim 1, wherein the subject is a mutant subject.

8. The method of claim 1, wherein the subject is a Drosophila.

9. The method of claim 8, wherein the Drosophila is a Drosophila melanogaster.

10. The method of claim 9, wherein the Drosophila melanogaster is w¹¹⁸.

11. The method of claim 8, wherein the Drosophila is a mutant Drosophila.

12. A method for identifying a molecular alteration in a subject comprising:
a. administering an inhibitor of histone deacetylase to the subject; and
b. identifying molecular alterations in the subject caused by said inhibitor of histone deacetylase by comparing the presence, level, and/or modification of nucleic acid and/or protein in the subject with the presence, level, and/or modification of nucleic acid and/or protein in a second subject that has not been administered an inhibitor of histone deacetylase, thereby indicative that the inhibitor of histone deacetylase induces a molecular alteration.

13. A method for identifying a test molecule that induces a molecular alteration in a subject, to which an inhibitor of histone deacetylase has been administered comprising:
a. administering a test molecule to the subject; and
b. identifying molecular alterations in the subject caused by said test molecule by comparing the presence, level, and/or modification of nucleic acid and/or protein in the subject with the presence, level, and/or modification of nucleic acid and/or protein in a second subject that has been administered an inhibitor of histone deacetylase, but was not administered with the test molecule, a difference in the presence, level, and/or modification of nucleic acid and/or protein thereby indicative that the test molecule induces a molecular alteration.

14. A method for identifying a molecule that extends the life span of a subject, wherein the subject has an extended life comprising:
a. administering a test molecule to the subject, to which an inhibitor of histone deacetylase has been administered;
b. comparing the life span of the subject of a) with a second subject, that has been administered an inhibitor of histone deacetylase and not administered the test molecule, a further extended life span by the subject of a) thereby identifies a molecule that extend the life span of the subject.

15. The method of claim 12 or 13, wherein the nucleic acid is a DNA or RNA.

16. The method of claim 15 wherein RNA is mRNA.

17. The method of claim 16, wherein the mRNA encodes a molecule selected from the group consisting of cytochrome P450, glutathione S-transferase 1-1, superoxide dismutase, transcription initiation factor TFIID 85kDa subunit, hepatocalcinoma-related transcription factor, daughterless protein, translation elongation factor 1alpha, translation initiation factor 4 gamma, ribosomal protein L9, ribosomal protein L10A, ribosomal protein L21, ribosomal protein S8, ribosomal protein S9, ribosomal protein S12, ribosomal protein S15A, ribosomal , protein S24, ribosomal protein S29, ribosomal protein PO, ribosomal protein P2, hsc70, hsp60, dnaJ like2, angiotensine-converting enzyme-like protein, aminopeptidase, aminopeptidase N, serine protease, serine protease, serine proteinase 2, angiotensine-converting enzyme precursor, stubble, serine proteinase, cysteine proteinase 1, leucine aminopeptidase, trypsin theta precursor, growth factor-regulated tyrosine , kinase substrate, guanine nucleotide-binding protein alpha, inactivation-no-afterpotential D, beta Adaptin (a), component of HA1 clathrin adaptor, guanyl-nucleotide exchange factor, epididymal secretory protein, SH2-SH3 adaptor protein, phosphorylase kinase gamma, p70-protein kinase(S6K), Fak like tyrosine kinase, Fps oncogene kinase, ADP/ATP translocase, mitochondrial phosphate carrier, sodium-dicarboxylate cotransporter, protein transport protein Sec23, neurotransmitter transporter, ADP/ATP translocase, transferrin precursor, putative odorant-binding protein A5 precursor, transportin, 26S proteasome subunit 4 ATPase, oxysterol-binding protein homolog Calphotin, T1/ST2 receptor binding protein precursor male specific protein, Neurocalcin homolog, ninaC, putative arginine-aspartate-rich RNA binding protein, TAR-binding protein, RNA binding protein, kurz protein, galactose-1-phosphate uridylyltransferase, mitochondrial aldehyde dehydrogenase, pyruvate kinase, aldehyde dehydrogenase 7, succinic semialdehyde dehydrogenase, citrate synthase, succinyl-CoA synthetase alpha subunit, dihydrolipoamide S-succinyltransferase, malate dehydrogenase, aspartate aminotransferase, serine-pyruvate aminotransferase 3-hydroxyisobutyrate dehydrogenase, 4-hydroxyphenylpyruvate dioxygenase, 4-amino butyrate amino transferase, haloacid dehalogenase-like hydrolase, phospholipase C, hydroxymethylglutaryl-CoA synthase, alpha esterase, 1-acyl-glycerol-3-phosphate acyltransferase, fatty acid desaturase, amidophosphoribosylamidotransferase, ATP synthase subunit g, ATP synthase subunit, vacuolar ATP, synthase subunit, Rho small GTPase, hook, myosin heavy chain, p47 protein, metasis-associated1-like protein, protein involved in sexual development, Cdc37, cell division cycle 37 protein, X-linked nuclear protein, microsomal epoxide hydrolase, imaginal disc growth factor 1, 18s rRNA, vitellogenin receptor, cystein proteinase 1, proteasome subunit, leucine aminopeptidase, mitochondrial processing protease-beta, ubiquitin conjugating enzyme, ribosomal protein S26, stubarista, ribosomal protein, dnaJ -1, guanine, nucleotide-binding protein gamma subunit, peroxisomal famesylated protein, midline fasciclin precursor, hexokinase, glyceraldehyde 3phosphate dehydrogenase 1, ATP synthase, subunit d, phosphogluconate, dehydrogenase, isocitrate dehydrogenase, aconitate hydratase precursor, acetyl-CoA carboxylase, hydroxyacyl-CoA dehydrogenase, AND-dependent 15-hydroxyprostaglandin dehydrogenase, fatty acid synthase, choline acetyltransferase, peptidyl gycine-alpha-hydroxylating monooxygenase, gamma-glutamylcysteine synthetase, tyrosine 3-monoxygenase, alpha-esterase, ATP synthase gamma, antennal-specific short-chain dehydrogenase/reductase, NADH:ubiquinone reductase 75kD subunit precursor, rhophilin, cytochrome c oxidase subunit Vib, cytochrome c oxidase, syntaxin, inorganic phosphate, cotransporter, tropomycine T, transferrin precursor, pheromone binding protein related protein 1 precursor, calreticulin, RNA helicase, osa, Cdk9, Mst87F, structural sperm protein, Transmembrane 4 Super Family, beta-spectrin, cut up, synaptogyrin homolog, tryptophanyl-tRNA synthetase, porin, and a voltage dependent anion-selective channel.

18. The method of claim 12 or 13, wherein the protein so modified is acetylated.

19. The method of claim 18, wherein the acetylated protein is a histone.

20. The method of claim 19, wherein the histone is methylated.

21. The method of claim 19, wherein the histone is H3.

22. The method of claim 19, wherein the histone is H4.
